(19) United States
(12) Patent Application Publication
(10) Pub. No.: US 2005/0009171 A1
(43) Pub. Date: Jan. 13, 2005

(54) Device and method for analyzing ion channels in membranes

(76) Inventors: Niels Fertig, Munchen (DE); Jan Behrends, Stegen (DE); Robert Blick, Munchen (DE)

Correspondence Address: IP Strategies, Suite 1, 12-1/2 Wall Street, Asheville, NC, 28801, US

(21) Appl. No.: 10/466,018

(22) PCT Filed: Jan. 7, 2002
(86) PCT No.: PCT/EP02/00078

(30) Foreign Application Priority Data
Jan. 8, 2001 (EP) 01100458.7

(51) Int. Cl. C12M 1/34

(52) U.S. Cl. 435/287.2

(57) ABSTRACT

The present invention relates to devices and methods for analyzing ion channels in membranes. The invention is characterized by a biochip comprising a substrate in which openings are provided in the form of an M×N matrix for receiving therein a cell membrane including at least one ion channel (I) or an artificial lipid membrane (Me), wherein M≧1 and N≧1.

### FIELD OF THE INVENTION

The present invention relates to devices and methods for analyzing ion channels in membranes, in particular devices and methods for executing the so-called patch clamp technique with the aid of a biochip, especially for use in high throughput processes.

### PRIOR ART

Ion channels are membrane proteins which serve as switchable pores for a flow of current. Ion channels, which are the smallest excitable biological structures, especially constitute the fundamental switching elements of the nervous system. It follows that the equipment of a neurocyte with ion channels of different types essentially determines the neurocyte's role in the processing of information in the brain. This applies, by the way, also to non-neuron excitable cells in a similar manner, e.g. to those of the cardiac muscle and its stimulus conduction systems. Switching processes in ion channels are analyzed for obtaining e.g. information on possible malfunctions and their elimination by means of drugs and the like.

For analyzing ion channels in cell membranes with respect to their switching processes, i.e. their opening and closing mechanisms, the patch clamp method is used in the prior art. For this purpose, so-called patch clamp pipettes consisting of glass are used. Such a pipette is shown in FIG. 5. This pipette comprises an opening 59 having a diameter of approx. 1 μm. In addition, the pipette comprises a pipette shaft 58 in which an electrode 53 is provided.

For analyzing an ion channel, a membrane patch is sucked up by means of such a pipette filled with an electrolyte so that a close contact will be established between the membrane and the glass. In this way, a very high sealing resistance of an order of magnitude of >1 GΩ is obtained. This permits measurement of very small ion currents, down to a few 100 fA, through the membrane.

The known device is, however, disadvantageous insofar as it is not suitable for simultaneously analyzing a large number of substances or the effect of a substance on a large number of different (e.g. genetically modified) ion channels. The known device is therefore not suitable for high throughput analyzing. Hence, this device is can be used for substance screening in the pharmaceutical industry only to a very limited extent.

Another disadvantage of the known device is that the time scale on which the opening and closing mechanisms in the ion channels take place is accessible only to a very limited extent with this device consisting of a glass pipette, an electrode and an amplifier. This has the effect that, when this device is used for the patch clamp method, the bandwidth will be limited to less than 100 kHz. For analyzing the opening and closing mechanisms in ion channels, time scales corresponding to a bandwidth of >1 MHz would, however, be desirable.

It is therefore the object of the present invention to provide a device for analyzing ion channels in cell membranes, which is suitable for high throughput processes, e.g. for use in the pharmaceutical industry, and/or which exhibits an improved signal-to-noise ratio and an improved timing resolution.

### DESCRIPTION OF THE INVENTION

This object is achieved by a biochip for analyzing ion channels, comprising a substrate in which openings are provided in the form of an M×N array for receiving therein a cell membrane including at least one ion channel or for receiving therein an artificial lipid membrane including at least one ion channel, wherein M≧1 and N≧1.

When such a biochip is used, the use of a pipette whose comparatively long shaft leads to a high stray capacitance can be dispensed with. The critical geometrical parameters can, however, be optimized from the very beginning, and this has the effect that the signal-to-noise ratio will be improved markedly in comparison with the prior art whereby the timing resolution will be improved. This applies to biochips with a single opening, i.e. for M=N=1 as well as to biochips with a plurality of openings, i.e. M>1 and/or N>1.

Due to the plurality of openings for receiving therein membranes including ion channels, it will additionally be possible to parallelize the patch clamp technique in the case of M>1 and/or N>1, whereby M×N measurements can be carried out simultaneously with one chip.

In this case it will be particularly advantageous to adapt the shape of this M×N array to the geometry of the 96, 384 or 1536 cuvette plates used as a standard in the pharmaceutical industry. These cuvette plates can be inserted into automatic pipetting devices by means of which substances can advantageously be applied to the biochip described here. A special advantage is that, by means of automatic pipetting devices or by other arrays of pipettes or cannulae which are arranged in a fixed mode relative to one another, solutions or cells can be taken simultaneously from a plurality of cuvettes of the standard cuvette plates and applied to the biochip, since the arrangement of the pipettes or cannulae relative to one another can be maintained for applying the solutions or cells to the biochip.

In addition, membranes which have been applied to the biochip according to the present invention will, in comparison with the known device, be much more easily accessible due to the geometry of the biochip. This offers a much better possibility of observing the membranes and of manipulating them chemically and/or mechanically and/or electrically.

According to an advantageous embodiment of the above-described biochip, the surface has in the area of each opening a means for improving the contact with the cell membrane, said means being provided on the receiving side of the respective opening and being used for guaranteeing improved adhesion of the membrane to the biochip in the area of the aperture (opening). Also the electrical sealing resistance can be increased in this way.

In accordance with an advantageous further development, the means for improving the contact can be implemented in the form of a patterning of the surface.

For this purpose, the patterning can be provided in the form of one or a plurality of rings which is or which are arranged around each opening, or in the form of one or a plurality of squares or rectangles which is or which are arranged around each opening.

The patterning can especially be provided in the form of a depression in the surface of the biochip, said depression being arranged concentrically around and in closely spaced relationship with the opening and having a diameter which is many times larger than the diameter of the opening so that the edge of the opening projects upwards beyond the surrounding biochip level. This has the effect that a cell membrane will be dented by the edge of the opening whereby the contact between the biochip and the membrane will be improved.

Each opening can have length and width dimensions in the range of 10 μm to 10 nm. The number of ion channels observed can be adjusted in this way. In addition, a smaller opening will also reduce the membrane area and thus the capacitance and this will improve the measurement resolution still further.

The biochip according to the present invention is also excellently suitable for forming artificial lipid membranes (artificial lipid bilayer) on the opening, this formation taking place in analogy with the known black lipid or lipid bilayer method. This permits ion channels to be analyzed by fusing vesicles, which include ion channels, with the artificial lipid bilayer.

Due to the fact that the size of the aperture is small in comparison with the known bilayer method (in known devices the size of the aperture is normally >100 μm) and due to the resultant small capacitance, the signal-to-noise ratio can be improved.

In accordance with a preferred embodiment of the above-described biochip, each opening can be substantially circular. Such circular shapes can easily be implemented in the bio-chip. If a simple implementation is not necessary, also other shapes can be chosen for the cross-sections of the openings.

According to a preferred further development of all the above-described biochips, the substrate can comprises a base portion which has a first thickness and a window portion or a plurality of window portions which is/are formed in said base portion and which has/have a second thickness, an opening being provided in each of the respective window portions. The thickness of the base portion can here especially range from 1 mm to 100 μm and the thickness of the window portion can range from 1 μm to 50 μm. This further development guarantees that the mechanical stability of the substrate will be preserved, whereas the length of the aperture (at right angles to the cross-section of the opening) and thus also the electric access resistance will remain as small as possible. In addition, this further development can be used for producing apertures with diameters of 10 μm down to less than 1 μm with the aid of a dry-etching step, laser ablation or latent ion track etching. On the basis of this further development it will also be possible to fill the aperture more easily with the electrolytic solution and to establish an electric contact therewith. The depression formed on the lower surface of the biochip by local thinning permits a simple application of solutions by means of a pipette; due to capillary forces, said solutions penetrate into the aperture and fill said aperture.

In accordance with an advantageous further development of all the above-described bio-chips, the substrate can comprise a semiconductor material, such as GaAs, Si or AlGaAs, or an insulator, such as glass or quartz, or polymers, such as polycarbonate, acrylic glass or polydimethylsiloxane (PDMS). A large number of advantages, in particular a simple production by means of a process technology perfected for the respective material, can be achieved by these materials.

According to an advantageous further development, the substrate comprising the base portion and the window portions formed in said base portion consists of one material. The production process of the biochip can be simplified in this way.

When a substrate consisting of a semiconductor material, in particular of Si, GaAs or AlGaAs, is used, a passivating and insulating layer can be provided, said layer being applied to one surface or to both surfaces of the substrate. This insulating layer can especially consist of SiO₂, Si₃N₄, glass or polymers, and of multi-layer systems in which these materials are combined with one another and/or with the above-mentioned semiconductors and/or with metals, and have thicknesses of 50 nm up to several μm. By means of these materials, a sealing resistance of a few GΩ can be realized, this kind of sealing resistance being necessary for measuring currents in the pA range.

In the production of this embodiment, the insulating layer can also fulfil the function of an etch stop layer and, in the case of anisotropic etching of the semiconductor it can lead to the formation of a window portion in which only the insulating layer is still present. The aperture can then be defined lithographically and the self-supporting insulating layer can be applied by dry-etching processes.

As a further advantageous alternative, polymers, such as polydimethylsiloxane (PDMS), can be used as a substrate material. When the above-described biochip is produced from PDMS, a 3D negative template (mould) is used, which has the inverted structure of the desired biochip. The PDMS is first viscous and, after having been mixed with a curing agent, it is cast into the mould and cured with or without heating (approx. 60 to 100 ° C.). The flexible biochip can then be released from the mould; said release can be carried out more easily when the mould has been coated with silanes previously. For the production of this embodiment a chemical modification of the surfaces (especially oxidation in the plasma incinerator or also other suitable methods) will be advantageous.

Moreover, all the surfaces of the biochip may be provided with additional insulating and passivating layers of the above-mentioned materials and they may have chemical modifications (silanization, oxidation).

According to a preferred further development of all the above-described biochips, electrodes can be provided on one or on both sides of the substrate. In particular, electrodes consisting e.g. of gold, silver or of other suitable metals can be applied directly to the chip by means of vapour deposition. This will simplify the test set-up, since the electrodes are already fixedly integrated on the biochip and since the step of applying and adjusting the electrodes can therefore be dispensed with. In addition, when this arrangement is used, and in particular when the electrodes are arranged such that the distance between said electrodes and the membrane is only a few μm, the parasitic capacitances and resistances can be reduced still further, and this will lead to another improvement of the signal-to-noise ratio.

Whether a biochip with integrated electrodes on one or on both sides of the substrate is used can be determined in dependence upon the test to be carried out. Electrodes which are particularly suitable for this purpose are Ag/AgCl electrodes. These electrodes have the advantage that an electrode polarization, which would corrupt the measurement results, will be avoided.

Furthermore, additional electrodes can be integrated so that high-frequency alternating electromagnetic fields can be applied via the aperture. In particular by applying a high-frequency alternating field in the range of MHz to GHz, the dynamics of the ion channels (conformation changes, ion permeation and ligand binding) can be influenced and analyzed. For applying such high-frequency fields, the use of antenna structures (e.g. the bow tie antenna known from the field of high-frequency technology) will be particularly suitable.

An effective coupling of the electromagnetic field to the ion channel can be achieved in this way. An advantageous alternative is the integration of planar waveguides (so-called strip lines) for high-frequency alternating fields.

The electrodes can have a width of 40 nm and they can be arranged at a distance of only a few nm from the opening so as to optimize coupling in of the power of the alternating fields.

When a substrate is used which comprises a base portion having a first thickness and one or a plurality of window portions formed in said base portion and having a second thickness, Ag/AgCl electrodes in the form of wires or sintered capsules (pellets) can be introduced in this recess, whereby the aperture will be electrically contacted as well.

For mechanically manipulating cells or liquids on the biochip, interdigital electrodes can be provided on the biochip for generating surface-acoustic waves with the aid of which cells or liquids can be positioned relative to the aperture of the biochip. In particular, surface acoustic waves can keep the cells in motion so that they will not adhere to the chip; this would make it impossible to suck them into the aperture or to cause them to move into said aperture in some other way.

According to a preferred further development of the above-described biochips, not only electrodes but also electrically and/or optically active and/or passive components can be integrated on the substrate. This results in a further structural simplification of the test set-up. Especially also the signal paths can be kept short in this way, and this will again have an advantageous effect on the signal-to-noise ratio. The biochips may, for example, comprise integrated field effect transistor means for preamplifying measuring signals.

The electrodes, the electrically and/or optically active and/or passive components can be integrated on the substrate in an advantageous manner, if desired on the etch stop layer and the insulating layer, respectively.

In accordance with further preferred embodiments, optical near-field means for observing the ion channel or the ion channels can be provided in all the above-described biochips. The possibility of using near-field means results from the geometry-dependent easy accessibility of a membrane on the biochip. Hence, especially all scanning probe methods, such as scanning force microscopy (AFM), scanning near-field optical microscopy (SNOM) and scanning tunneling microscopy (STM), can be used easily for observing the membranes.

On the basis of the geometry-dependent easy accessibility, also other image-forming methods, such as scanning electron microscopy (REM), confocal fluorescence microscopy (also in combination with SNOM), fluorescence spectroscopy, optical microscopy or individual photon detection, can be used. In particular biochips consisting of glass or polydimethylsiloxane (PDMS) are suitable for fluorescence tests, since the substrate has here a weak fluorescent background.

In accordance with an advantageous embodiment, microfluid channels can be provided in the above-described biochips for on-chip perfusion.

According to a particularly advantageous further development of all the hitherto described biochips, the biochip has applied thereto a layer of flexible, non-conductive polymer on the receiving side, said layer comprising at least two openings through which at least the openings in the substrate are exposed. It follows that the area of an opening in the polymer layer is at least as large as the area of an opening in the substrate. The layer is preferably 10 μm to 5 mm thick and consists e.g. of PDMS. The openings may, for example, be produced by punching. Through these openings in the flexible polymer, whose diameter can be e.g. 10-5000 μm, individual areas resembling cuvettes are defined on the biochip on the receiving side; these cuvette-like areas serve to receive liquid therein and the substrate of the biochip including at least one aperture is exposed in said areas on the receiving side. A particularly advantageous aspect of this arrangement is that the individual apertures are thus also electrically separated from one another on the receiving side. Each opening in the polymer layer may, for example, expose precisely one aperture and part of the substrate surrounding said aperture. Alternatively, also a plurality of apertures can be exposed by on opening in the polymer layer; in this case, a cuvette encloses a plurality of apertures. PDMS is particularly suitable as a substrate for these cuvettes, since it has good adhesive properties with respect to glass and quartz as well as with respect to the other above-mentioned substrates which can be used for designing the biochip, and since it is biocompatible.

Alternatively, the substrate surface of the biochip can be rendered hydrophobic by treatment with chemicals so that solution drops deposited on the receiving side on top of the apertures will rest on said apertures with a steep contact angle and remain reliably separated from one another. This has the effect that, without the aid of any additional structure a liquid compartment will be formed, which is effective as a cuvette as well.

According to another particularly advantageous embodiment of all the above-described bio-chips, channels extending parallel to the substrate surface are provided in or above said substrate surface. Alternatively, these channels are formed directly as trenches in the surface of the substrate and are open at the top. According to another advantageous alternative, the biochip is, on the receiving side, provided with a PDMS layer or any other substrate which is adherent to the biochip and through which trenches extend that are open towards the surface of the biochip substrate including the aperture. These trenches may especially have diameters and depths between 5 and 500 μm. By applying the layer containing these trenches to the biochip, said trenches become fluid channels which are closed by the substrate surface of the biochip. In accordance with a specially preferred embodiment, these trenches are designed in such a way that they extend in a cross-shaped or star-shaped pattern towards and away from the apertures. In accordance with a specially preferred embodiment of this further development, these channels are furthermore dimensioned such that cells contained in a liquid flowing through said channels will move either individually (one after the other) or in some other arrangement through said channels. Hence, such channels are suitable for moving cells horizontally to the chip surface from the periphery of the biochip accurately over and across the apertures in such a way that, when a vacuum is applied through an aperture, this will immediately have the effect that the respective cell on top of said aperture will be sucked in.

The above-described biochips can be produced in a simple way. Fundamentally, the following steps are common to all methods: providing a substrate, forming one or a plurality of window portions in said substrate, and forming one opening per window portion.

In the case of a biochip on the basis of a semiconductor substrate with an insulating layer, it will be advantageous to use the following method for forming the window portion: an insulating layer, which is provided on the upper and on the lower side and which is resistant to the wet-chemical etching method (especially KOH), is removed on the lower side in a lithographically defined area by a dry-etching step, whereby the semiconductor substrate will be exposed directly in this area. The following wet-chemical etch step (especially KOH) then causes, by anisotropic etching, the formation of an etch trench having the form of an inverse pyramid. If the primary exposed substrate surface is sufficiently large, this etch trench can extend up to the opposite side, but due to the insulating layer provided on said opposite side, which is resistant to the wet-chemical etchant and acts therefore as an etch-stop layer, the trench will remain closed on one side in any case. This permits a precise implementation of a rectangular window portion in a very simple manner, the area of said window portion depending on the area of the substrate exposed on the lower side in the first step. Layers which proved to be advantageous as an etch-stop or insulating layer are especially an Si₃Nₓ layer, preferably an Si₃N₄ layer, an SiO₂ layer, or Si₃Nₓ/SiO₂ multi-layer systems.

Finally, the opening itself can be formed in the window portion by optical lithography and a dry-etching step. This method is suitable for comparatively large openings (≧1 μm). If smaller openings, i.e. openings down to a size of 10 nm, are to be provided, the opening can be formed e.g. by electron-beam lithography and a dry-etching step. According to a preferred alternative, the opening can be formed by means of a focussed ion beam.

When the biochip is implemented on a glass substrate or on a quartz substrate, an isotropic HF etching method can be used for defining the window portion by local thinning of the glass substrate. Likewise, the window portion can alternatively be formed by ablation with a laser having a suitable wavelength or by hot shaping (hot pressing).

The actual opening can be formed in the window by lithography in combination with a dry-etching step on the one hand. In the case of these substrate materials, the aperture can also be produced by etching by means of a latent track of a single high-energy ion which has passed through the thinned window area. On the other hand, it also possible to form, according to a preferred embodiment, the aperture in the thinned window portion by ablation with a laser having a suitable wavelength. For this purpose, it will be particularly advantageous to use an excimer laser having a wavelength in the ultraviolet region. Especially when the substrate in the window portion has previously been thinned to a thickness between 10 and 50 μm, apertures having a diameter of less than 10 μm down to less than 1 μm can be produced by irradiation with laser light.

According to a preferred embodiment of all the above-described biochips, the substrate surface, the edge of the aperture or the inner wall of the aperture can be treated by local heating, e.g. by a laser having a suitable wavelength, (so-called tempering), so as to make said substrate surface, said edge of the aperture or said inner wall of the aperture more suitable for close contact with a cell membrane, smooth them, by way of example, or modify the chemical structure of the substrate in a suitable way. This can also be done by non-local heating of the whole biochip. The temperatures reached during local or non-local heating may be lower as well as higher than the melting point of the respective substrate.

When the biochip is made from PDMS no etch step will be carried out, since a moulding process is here used, i.e. the window portion as well as the openings are transferred from a 3D negative template. The etching methods and the lithography methods described are, however, used for producing the negative template.

All the above-described advantageous further developments can be used for biochips with an opening (M=N=1) as well as for biochips with a plurality of openings (M>1 and/or N>1).

All the above-described biochips can be used not only for the conventional analyzation of ion channels in membranes but also for a great variety of other purposes.

The opening or the openings of the biochip can have incorporated therein subareas of the cell membrane of cells (e.g. cells isolated from tissues or primary cultures, and cell lines, which express certain ion channels). For this purpose, it will be advantageous to position first one cell per aperture. In order to do so, singulated (non-coherent) cells in an aqueous suspension are applied to the biochip, the aperture being already filled with an electrolytic solution.

According to an advantageous embodiment, cells are applied with the aid of at least one pipette or cannula. This can be done automatically, e.g. by means of electronically controlled xyz motors. In a preferred embodiment, a separate pipette or cannula is provided for each aperture.

According to a further particularly advantageous arrangement, these pipettes or cannulae include integrated electrodes which are suitable for measuring the ion current through ion channels and which are in electric contact with the cuvette and consequently the aperture via the electrolytic solution contained in the pipette or cannula. Providing such measuring electrodes on the chip substrate on the receiving side is then no longer necessary.

If the biochip is provided with channels extending parallel to the substrate surface, as has been described hereinbefore, one or a plurality of singulated cells can be flushed into the biochip through these channels where they can be positioned on a respective opening.

For positioning a cell on the aperture, a vacuum can be applied from the aperture side located opposite the receiving side so that the resultant flow of fluid will move a cell onto the aperture. Alternatively or additionally a constant electric field can be applied via the aperture. This will promote the formation of a tight contact between cell and biochip.

Again alternatively or additionally, direct voltage or alternating voltage fields can be applied through suitable electrodes provided on the biochip; by means of these voltage fields, cells are electrophoretically or dielectrophoretically moved towards the aperture or held in position on said aperture.

Again alternatively or additionally, surface-acoustic waves produced by further electrodes can be used for positioning cells or liquid drops containing cells on the aperture.

Again alternatively or additionally, further mechanical, chemical (e.g. osmotic or oncotic), electric, magnetic or electromechanical gradients or fields can be applied through the aperture so as to move cells directly or indirectly towards the aperture.

Alternatively or additionally, further cells or other particles or solutions can be added on the receiving side so as to position cells on the aperture; due to their specific weight or due to other properties, these further cells or particles or solutions will move the cells mechanically and/or by other forces towards the receiving-side surface of the biochip and or towards the aperture and/or fix them there.

Preferably, all the above-described methods for positioning a cell on an aperture are also used for fixing the cell on the aperture.

Preferably, an electrophysiological characterization of each cell is carried out by means of the above-described biochips.

In analogy with the so-called whole-cell voltage clamp known from the field of patch clamp technology, it is also possible to establish contact with the interior of a whole cell via the aperture. It will be advantageous to do this by abruptly reducing the pressure in the aperture (suction pulse) for a short time (duration: preferably 10 ms to 10 s, amplitude: preferably −10 to −1000 mmHg), by applying an electric voltage pulse (duration: preferably 0.1 to 1000 ms, amplitude preferably 100 mV to 10 V) or by adding a pore-forming agent (e.g. gramicidin or nystatin) for perforating the membrane portion located in the aperture.

The presence of a cell on top of the aperture can be detected by measuring the conductance or the high-frequency impedance or other electric parameters of the aperture. Subsequently, the suction pulse can be triggered, for example.

According to an advantageous embodiment, an application or de-application of active substances is carried out by flushing in or sucking off a solution. Flushing in or sucking off can be effected by pipettes or cannulae. If fluid channels exist, they can be used for flushing in or sucking off. The application or de-application of active agents can take place prior to or during a measurement.

Furthermore, all the above-described biochips can be provided with devices which are arranged on the lower side located opposite the receiving side and which permit the simple application of a negative pressure or of an excess pressure relative to the upper side (i.e. a pressure gradient through the apertures). These devices can be implemented e.g. as hollow chambers in a flexible polymer substrate (e.g. PDMS), which are filled with liquid and which are located below each of the respective openings and window portions; these hollow chambers are connected to the respective apertures and through said apertures with the upper side of the biochip and their volume can be reduced in size by pressure applied from outside and generated by a mechanical device, and re-enlarged by reducing said pressure.

The application of a pressure gradient through the apertures can also take place through micro-fluid channels and hose systems communicating with these channels.

According to another preferred embodiment, one of the biochips described can also be combined with a further second biochip provided with a means for positioning cells relative to the openings of said first biochip, the respective surfaces on the receiving side being located in opposed relationship with and at a fixed or variable distance from one another. This combination can be established e.g. by a fixed or a flexible connection of the two biochips in such a way that their respective receiving-side surfaces are opposed to one another and are e.g. either separated by a gap of 10-1000 μm width or in direct contact with each other. It will be advantageous when the means for positioning cells of the second biochip comprises a means for generating surface waves. If the biochips are supported in direct contact with each other, the receiving-side surface of the second biochip is provided with fluid channels which extend preferably parallel to the surface and which are open towards the surface. In this case, the cells can be flushed in through these fluid channels.

The biochips according to the present invention can also be used in a measuring probe comprising a glass tube provided on the side of the substrate which is located opposite to the side where the membrane is applicable, the opening of the glass tube facing away from the substrate being implemented such that an electrode can be inserted therein. This offers the possibility of moving an electrode in an ionic solution towards the opening for analyzing the ion channel or the ion channels.

Alternatively, a holding device consisting of polycarbonate or of some other material apart from glass can be provided instead of a glass tube; this holding device can be provided with a central cavity or a plurality of cavities, which communicate with the aperture or the apertures of the biochip and to which the biochip is adhesively attached or fixed in some other way, an electrode or a plurality of electrodes in an ionic solution being adapted to be inserted in said holding device. At these cavities communicating with the apertures of the biochip, means can again be provided, which permit the application of an excess pressure or of a negative pressure so that cells originating from a suspension applied on the receiving side can be kept away from or sucked into the aperture. In particular, the biochip and the device including the cavities can have provided between them a layer of a flexible polymer substrate (e.g. PBMS) so as to guarantee a tight seal.

In this way, a means is obtained in which the above described biochip can easily be integrated. Especially, it will also easily be possible to integrate this measuring probe in known patch clamp set-ups, especially in upright and inverted optical microscopes and measurement sites for optical and mechanical scanning probe methods.

In accordance with an advantageous embodiment, the opening of the glass tube or of the holding device facing away from the substrate can, in such a measuring probe, be implemented such that an electrode means can be screwed into said opening. In this kind of arrangement, the electrode means can be replaced rapidly and can, moreover, be reused. This arrangement is suitable for use e.g. with a biochip having integrated electrodes only on the upper side of the chip.

The measuring probe can, in an expedient manner, also be sold together with the screw-in electrode.

In such an arrangement it will be expedient to provide sealing means, e.g. O-rings, between the opening of the glass tube and the screw-in electrode so as to retain the electrolyte in the glass tube or holding device.

According to an advantageous embodiment, the glass tube or the holding device is adapted to be adhesively attached to the substrate or to be screw-fastened to the substrate making use of a sealing ring. A simple and tight connection between the glass tube and the substrate can be guaranteed in this way. Fastening by means of screwing according to the second alternative additionally leads to a simple re-usability of the biochip, since it permits aggressive cleaning of the biochip.

The above-described measuring probes can advantageously be implemented in such a way that they comprise a means for generating a vacuum in the glass tube or the holding device. With the aid of this means, a membrane patch of a cell, which is also in solution, can be defined by the usual suction technique. This means that all the steps required for carrying out an analysis of ion channels can be executed at a single device. This leads to improved handling properties of the device.

In the following, special embodiments of the present invention will be explained making reference to the drawing enclosed, in which:

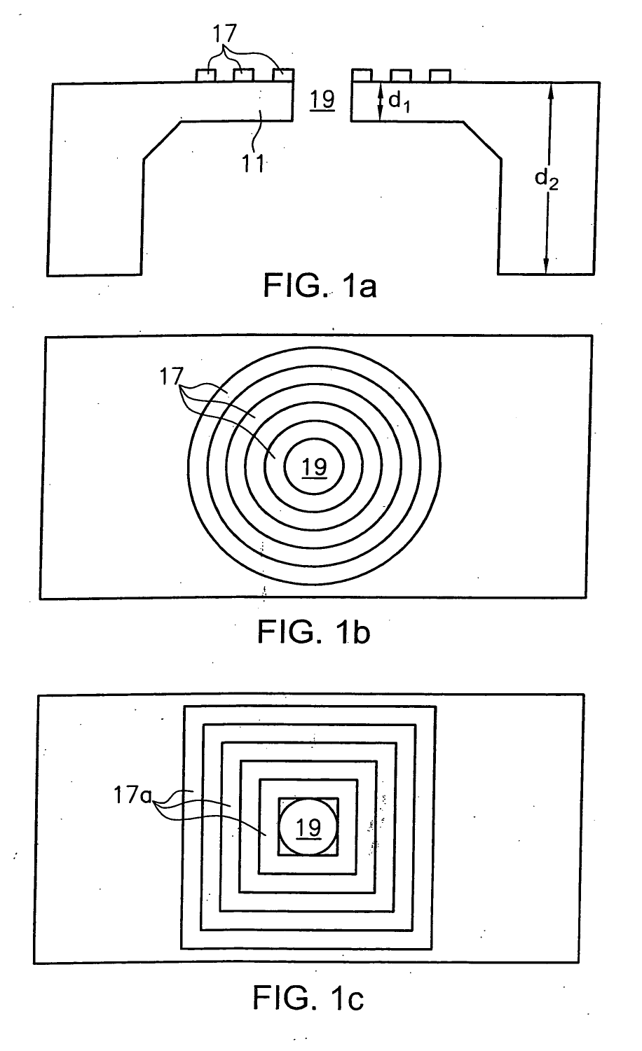

FIG. 1a shows a sectional view of a first embodiment of a biochip according to the present invention;

FIG. 1b shows a top view of said first embodiment of a biochip according to the present invention;

FIG. 1c shows a top view of a modification of the first embodiment of a biochip according to the present invention;

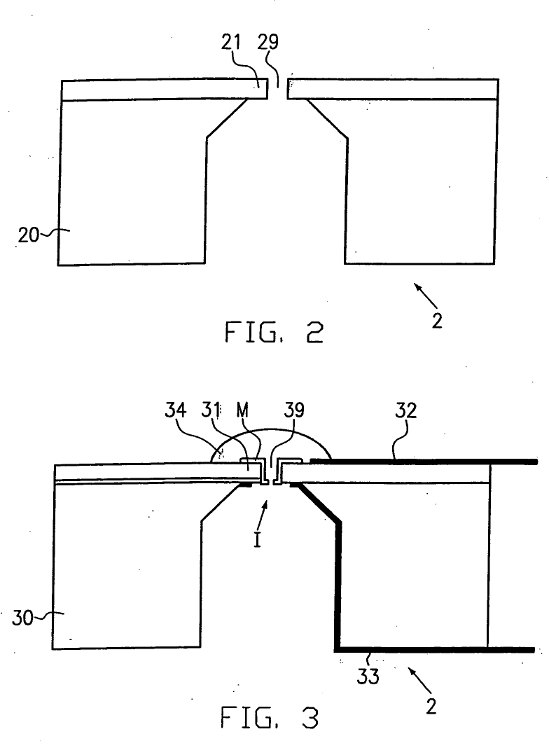

FIG. 2 shows a second embodiment of the biochip according to the present invention;

FIG. 3 shows a third embodiment of the biochip according to the present invention;

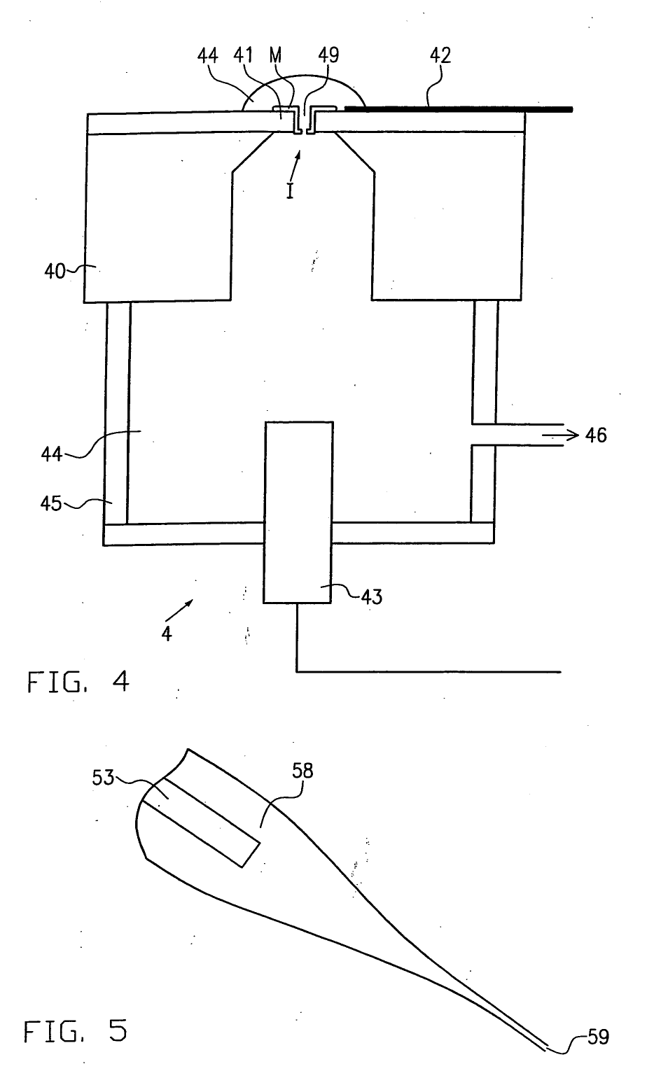

FIG. 4 shows an embodiment of the measuring probe according to the present invention; and

FIG. 5 shows a pipette for analyzing ion channels according to the prior art.

FIG. 1a and 1b show a first embodiment 1 of a biochip according to the present invention.

This biochip comprises a substrate formed with an opening 19 for receiving therein a cell membrane which comprises at least one ion channel. In the present case, the biochip is shown with M=N=1.

The substrate comprises a base portion 10 with a first thickness d, and a window portion 11 with a second thickness d₂ in which the opening 19 is provided.

The thickness of the base portion 10 ranges from 1 mm to 100 μm and the thickness of the window portion ranges from 1 μm to 50 nm. The window portion has an area of a few 10 μm² to 0.1 mm².

The opening 19 is substantially circular and has a diameter which ranges from 10 μm to 10 nm. The size of the opening is determined by the number of ion channels which are to be analyzed in a cell membrane.

The biochip 1 consists of a (0001) quartz (Z cut) in which the window portion 11 is first formed by an anisotropic wet-chemical etch step. The etchant used for this purpose is HF.

Depending on the size of the desired opening, said opening is formed in the last step by optical lithography and a dry-etching step or by electron-beam lithography and a dry-etching step.

Furthermore, the surface of the biochip according to FIG. 1 is, in the area of the opening, provided with a means for improving the contact between the biochip and the cell membrane. In the present case, this means is formed by patterning the surface. For this purpose, annular raised portions 15 are provided, which are arranged around the opening.

These raised portions have the effect that the membrane with an ion channel to be analyzed will be dented, whereby improved adhesion will be achieved due to a hydraulic effect and the electrical sealing resistance will be increased.

The patterning in the biochip according to FIGS. 1a and 1b is only exemplary. It is especially also possible to use other forms of raised portions, e.g. one or a plurality of squares or rectangles, which is or which are arranged around each opening. One of these alternatives is shown in FIG. 1c.

FIG. 2 shows a second embodiment of a biochip according to the present invention.

Also this biochip comprises a substrate 20, 21 formed with an opening 29 for receiving therein a cell membrane which comprises at least one ion channel. Also in the case of the biochip shown in FIG. 2, M=N=1.

The geometrical shape and the dimensions of the biochip 2 correspond to those of the biochip 1 shown in FIG. 1. In order to avoid repetitions, reference is only made to the relevant description of FIG. 1 in this connection. The reference numerals of corresponding parts differ from one another only with respect to their first figure.

The substrate of the biochip 2 comprises a base portion 20, which is again made from quartz, and an etch-stop layer in which the window portion 21 is formed. This etch-stop layer consists of Si₃Nₓ, preferably of Si₃N₄.

A characteristic feature of biochip 2, in comparison with biochip 1 of FIG. 1, is that it can be produced by a simplified method.

The substrate 20 has first applied thereto an etch-stop film. Subsequently, the window portion 21 is formed from the opposite side up to said etch-stop layer, said window portion being formed by an anisotropic wet-chemical HF etch step. Finally, the opening is formed preferably by one of the methods described in connection with the first embodiment.

FIG. 3 shows a first embodiment of a biochip 3 according to the present invention.

With regard to the geometrical dimensions and the structural design, the biochip 3 essentially corresponds to the structural design of the biochips described in FIGS. 1 and 2 so that reference is here once more made to the description of these chips in order to avoid repetitions. The reference numerals of corresponding parts differ from one another only with respect to their first figure.

In contrast to the biochips shown in these FIGS. 1 and 2, the base portion 30 of the substrate consists of a semiconductor material, e.g. (100)-Si.

This semiconductor material has applied thereto an insulating layer in which the window portion 31 is formed. The insulating layer 31 additionally serves as an etch-stop layer in the production process.

The production process is therefore similar to that of the biochip produced according to FIG. 2. In the embodiment shown, this layer consists of Si₃N₄.

In particular, the insulating and etch-stop layer is first applied to the silicon base portion 30 by means of a PECVD method. Following this, the window portion 31 is formed in the substrate from the opposite side, said window portion being formed by an anisotropic wet-chemical KOH etch step. In so doing, etching is executed up to the etch-stop layer. Depending on the desired size of the opening, said opening can then be formed, in a manner corresponding to the above-described embodiments, by optical lithography or electron-beam lithography and a dry-etching step.

In the last step, the electrodes 32 and 33, which consist here of Ag/AgCl, are applied to the upper and to the lower surface of the substrate.

In FIG. 3 it is also shown how a membrane Me with an ion channel I has been introduced in the opening 39. For the subsequent measurement, which will be described in detail with reference to FIG. 4, an electrolytic liquid 34 must be provided on top of the membrane and the electrode 32 as well as in the etch trench.

FIG. 1 to 3 each show biochips with M=N=1. It goes without saying that the statements made hereinbefore also apply to biochips with substrates in which a plurality of openings is provided. These openings can be provided in the form of an M×N array. In such an array they can be arranged regularly or such that the individual rows are displaced relative to one another.

The biochips shown in FIG. 1 to 3 only represent preferred embodiments of the present invention and should not be regarded as a limitation of said invention.

Hence, a large number of other embodiments, which are not shown, is possible.

It is, for example, not necessary that the opening is circular. It may have different cross-sections, depending on the respective requirements to be satisfied.

In addition, various materials can be used for forming the biochips. It will, for example, be possible to use glass instead of the quartz, and, instead of the silicon, a different semiconductor material, e.g. GaAs, may be used.

Especially in the case of a substrate consisting of a semiconductor material, but not exclusively in the case of such substrates, the surfaces of the substrate may be coated with a passivating layer.

Furthermore, different kinds of electrodes can be used, e.g. electrodes which are suitable for generating an electromagnetic field in the area of the ion channel.

In addition, electrically and/or optically active and/or passive components can be integrated. on the substrate.

Likewise, various methods which are known to a sufficient extent from the field of semiconductor technology can, in dependence upon the respective materials used, be employed for producing the biochips.

FIG. 4 shows a measuring probe according to one embodiment of the present invention.

This measuring probe includes a substrate comprising a base portion 40 and a window portion 41 in which an opening 49 is formed. In addition, a first electrode 42 is arranged on the substrate.

Below the substrate 40, a holding device 45 is secured in position, which is provided with a central cavity communicating with the opening 49 and which is followed by an electrode 43 with a holder.

In addition, the measuring probe comprises a means for generating a vacuum in the holding device, said means being designated by reference numeral 46.

In addition to the embodiment of the measuring probe which is shown here and which should. not be regarded as a limitation of the present invention, further modifications are possible.

For example, arbitrary ones of the biochips according to the present invention can be used as biochips. In particular the dimensions are then determined by the respective field of use, i.e. especially by the number of the channels to be analyzed.

The holding device may e.g. secured to the substrate by means of an adhesive.

The electrode means including the holder can be implemented such that it can be screwed into the holding device from below.

Furthermore, a sealing ring can be provided between the opening of the holding device and the electrode that can be screwed in.

In the following, it will be described how ion currents through the ion channel can be measured by the present measuring probe.

The cell membrane is first applied to the substrate in an electrolytic solution. By actuating the vacuum generating means 46, the membrane including the ion channel is sucked into the opening. The measuring probe contains an electrolytic solution 44 as well. Finally, the current flowing through the ion channel can be measured via the two electrodes 42 and 43.

1. A biochip (1; 2; 3) for analyzing ion channels, comprising a substrate (10; 20; 30) in which openings (19; 29; 39) are provided in the form of an M×N matrix for receiving therein a cell membrane (Me) including at least one ion channel (1) or an artificial lipid membrane including at least one ion channel, wherein M≧1 and N≧1.

2. A biochip (1; 2; 3) according to claim 1, wherein the surface of the biochip has in the area of each opening a means for improving the contact between the cell membrane and the biochip, said means being provided on the receiving side.

3. A biochip according to claim 2, wherein the means for improving the contact is implemented in the form of a patterning of the surface.

4. A biochip according to claim 3, wherein said patterning is provided in the form of one or a plurality of rings which is or which are arranged around each opening, or in the form of one or a plurality of squares or rectangles which is or which are arranged around each opening.

5. A biochip according to claim 1, wherein each opening is substantially circular.

6. A biochip according to claim 1, wherein the substrate comprises a base portion (10; 20; 30) which has a first thickness (d₁) and window portions (11; 21; 31) which are formed in said base portion and which have a second thickness (d₂), each opening being provided in a respective window portion.

7. A biochip according to claim 1, wherein the substrate comprises a semiconductor material, such as GaAs, Si or AlGaAs, or an insulator, such as a glass or quartz, or polymers, such as polydimethyl-siloxane (PDMS).

8. A biochip according to claim 6, wherein the substrate comprising the base portion and the window portions formed in said base portion consists of one material.

9. A biochip according to claim 1, wherein electrodes are provided on one or on both sides of the substrate.

10. A biochip according to claim 9, wherein the electrodes are implemented such that they are adapted to have applied thereto a temporally constant electromagnetic field and/or a high-frequency alternating electromagnetic field.

11. A biochip according to claim 1, wherein planar waveguides are integrated in the biochip for applying high-frequency alternating fields.

12. A biochip according to claim 1, wherein interdigital electrodes are provided on the biochip for generating surface-acoustic waves.

13. A biochip according to claim 1, wherein active and/or passive components are integrated on the substrate.

14. A biochip according to claim 13, wherein said active and/or passive components comprise a field effect amplifier means for preamplifying measuring signals.

15. A biochip according to claim 1, wherein an optical near-field means is provided for observing the ion channel or the ion channels.

16. A biochip according to claim 15, wherein the optical near-field means comprise scanning probe means.

17. A biochip according to claim 1, wherein microfluid channels are provided for on-chip perfusion.

18. A biochip according to claim 1, wherein the biochip has applied thereto a layer of flexible, non-conductive polymer on the receiving side, said layer comprising at least two openings through which at least the openings in the substrate are exposed.

19. A biochip according to claim 1, wherein the surface on the receiving side is hydrophobic.

20. A biochip according to claim 1, wherein channels extending parallel to the substrate surface are provided in or above said substrate surface.

21. A method of producing a biochip for analyzing ion channels comprising a substrate in which openings are formed, in the form of an M×N matrix, for receiving therein a cell membrane including at least one ion channel or an artificial lipid membrane including at least one ion channel, wherein M≧1 and N≧1, said method comprising the steps of:
providing a substrate,
forming at least one window portion in said substrate, and
forming an opening in each window portion.

22. A method according to claim 21, wherein each window portion is formed by means of wet- or dry-etching methods.

23. A method according to claim 21, wherein each window portion is formed by means of laser thinning or by means of hot shaping.

24. A method according to claim 21, wherein each opening is formed by means of laser thinning or ion track etching.

25. A method according to claim 21, wherein each opening is formed by means of dry-etching methods or by means of a focused ion beam.

26. A method according to claim 21, comprising the following additional step:
local or non-local heat treatment of the substrate for improving the contact with a cell membrane.

27. A method of analyzing ion channels in membranes, said method comprising the steps of:
providing a biochip according to claim 1,
applying one or a plurality of singulated cells in an aqueous suspension to the biochip,
positioning not more than one cell on one opening.

28. A method according to claim 27, wherein the cells are applied with the aid of at least one pipette or cannula.

29. A method according to claim 28, wherein the ion channel currents are measured with the aid of electrodes integrated in each pipette or cannula.

30. A method of analyzing ion channels in membranes, said method comprising the steps of:
providing a biochip according to claim 20,
flushing one or a plurality of singulated cells in an aqueous suspension into the biochip via the channels extending parallel to the substrate surface,
positioning not more than one cell on one opening.

31. A method according to claim 27, wherein, for positioning each cell, a vacuum is applied at the side of an opening located opposite the receiving side.

32. A method according to claim 27, wherein, for positioning each cell, an electric direct voltage and/or alternating voltage is/are applied perpendicularly to the substrate surface.

33. A method according to claim 27, wherein surface-acoustic waves are used for positioning each cell.

34. A method according to claim 27, wherein, for positioning each cell, mechanical, chemical, electric, magnetic or electromechanical gradients or fields are applied through the opening.

35. A method according to claim 27, wherein, for positioning each cell, additional cells or particles are added on the receiving side.

36. A method according to claim 27, said method comprising the following additional step:
detecting each cell on an opening by measuring at least one electric parameter of said opening.

37. A method according to claim 27, said method comprising the following additional step:
electrophysiological characterization of each cell.

38. A method according to claim 27, wherein active substances are applied or de-applied by flushing in or sucking off a solution.

39. A device for analyzing ion channels in membranes, comprising:
a first biochip according to claim 1, and
a second biochip provided with a means for positioning cells relative to the openings of said first biochip,
wherein the respective surfaces on the receiving side are located in opposed relationship with and at a fixed or variable distance from one another.

40. device according to claim 39, wherein the cell positioning means comprises a means for generating surface waves.

41. A device according to claim 39, wherein the biochips are supported in direct contact with one another and wherein the surface of the second biochip located on the receiving side has integrated therein fluid channels which extend parallel to the surface and which are open towards said surface.

42. A measuring probe (4), comprising
a biochip (1; 2; 3) according to claim 1,
a holding device (45) having a central cavity or a plurality of cavities which communicate with the aperture or the apertures of the biochip (1; 2; 3) and provided on the side of the substrate that is located opposite to the side where the membrane (M) is applicable, wherein
the opening of the holding device facing away from the substrate is implemented such that an electrode means (43) can be inserted therein.

43. A measuring probe according to claim 42, wherein the holding device consists of glass or polycarbonate.

44. A measuring probe according to claim 42, wherein the holding device is adapted to be screw-fastened to the biochip.

45. A measuring probe according to claim 42, wherein sealing means are provided between the holding device and the biochip.

46. A measuring probe according to claim 42, wherein the holding device is adhesively attached to the substrate.

47. A measuring probe according to claim 42, wherein the electrode means is adapted to be screwed into the glass tube.

48. A measuring probe according to claim 47, wherein sealing means are provided between the holding device and the electrode means.

49. A measuring probe according to claim 42, wherein a vacuum-generating means (46) is provided in said glass tube.
